(19) United States
(12) Patent Application Publication
(10) Pub. No.: US 2021/0086266 A1
(43) Pub. Date: Mar. 25, 2021

(54) ADDITIVE MANUFACTURING COMPONENTS AND METHODS

(71) Applicant: The University of Liverpool, Liverpool (GB)

(72) Inventors: Kate Black, Liverpool (GB); Paul Chalker, Liverpool (GB); Christopher John Sutcliffe, Liverpool (GB)

(73) Assignee: The University of Liverpool, Liverpool (GB)

(21) Appl. No.: 16/635,435

(22) PCT Filed: Aug. 1, 2018
(86) PCT No.: PCT/GB2018/052205

§ 371 Date: Jan. 30, 2020

(30) Foreign Application Priority Data
Aug. 1, 2017 (GB) 1712384.5

(51) Int. Cl. B22F 10/14; B22F 10/64; B33Y 10/00; B33Y 30/00; B33Y 70/00; B33Y 40/20; B28B 1/00; B29C 64/165; B22F 1/00; C04B 35/628; A61L 27/04; A61L 27/10; A61L 27/42

(52) U.S. Cl. CPC B22F 10/14; C04B 2235/6026; B33Y 10/00; B33Y 30/00; B33Y 70/00; B33Y 40/20; B28B 1/001; B29C 64/165; B22F 1/0011; B22F 1/0018; B22F 1/0059; C04B 35/62802; A61L 27/047; A61L 27/10; A61L 27/427; B22F 2301/205; B22F 2301/15; B22F 2301/10; B22F 2304/10; B22F 2304/054; C04B 2235/5454; C04B 2235/5445; C04B 2235/5436; B22F 10/64

(57) ABSTRACT

A method of 3D printing comprises: providing a layer of a powder bed; jetting a functional binder onto selected parts of said layer, wherein said binder infiltrates into pores in the powder bed and locally fuses particles of the powder bed in situ; sequentially repeating said steps of applying a layer of powder on top and selectively jetting functional binder, multiple times, to provide a powder bed bonded at selected locations by printed functional binder; and taking the resultant bound 3D structure out of the powder bed.

### FIELD OF THE INVENTION

The present invention relates to additive manufacturing, also known as 3D printing, and in particular to binder jetting, components used in binder jetting, and resultant products.

### BACKGROUND TO THE INVENTION

Additive manufacturing, commonly referred to as 3D printing, is a term which encompasses several categories of processes by which 3D objects are formed or “printed”. The 3D objects are generally built up layer by layer, and the processes differ in the way that the layers are formed and in what they are made from.

Some processes entail polymerising or curing liquid material. For example, in vat photopolymerisation, a platform is lowered into a vat of liquid polymerisable material (e.g. epoxy acrylate resin) so that it is slightly below the surface. Laser radiation is used to polymerise and harden selective parts of the layer above the platform. The platform is then lowered slightly so that a new liquid layer is at the surface (this may be made uniform by using a levelling or coating blade) and the polymerisation process is repeated. This procedure of lowering, coating and polymerising is repeated layer by layer until the desired three-dimensional structure has been formed. The platform may then be raised and the product removed and processed further. Post-processing typically involves the removal of support structures (which may be formed during the polymerisation steps) and any other residual material, and then high temperature curing following by finishing, e.g. sanding of the product.

Some other processes entail forming each layer of a 3D structure by extruding a plastic or polymer material (or, less commonly, other material). This is known as extrusion deposition or fused deposition modelling (FDM). Material, e.g. a polylactic acid resin, is fed to an extruder where it is heated and extruded through a nozzle which moves in X and Y directions. The selectively deposited material solidifies on cooling. As with vat polymerisation methods, the structure usually rests on a build platform which typically moves downwards between the deposition of each layer, and support structures are typically required, particularly for overhanging parts of structures. Such extrusion methods are amongst the most common 3D printing processes and used widely in consumer 3D printers.

Another category of additive manufacturing is material jetting which is similar to extrusion deposition in that material is deposited via a nozzle which moves in X and Y directions. Instead of being extruded, the material is jetted onto a platform. The material (e.g. wax or polymer) is applied as droplets using a print head, similar to conventional two-dimensional inkjet printing. The droplets solidify and then successive layers are applied. Once the structure is formed it may be subjected to curing and post-processing. As with other methods discussed above, support structures may be incorporated during the procedure and then removed during post-processing.

Powder bed fusion (PBF) methods entail the selective binding of granular materials. This can be done by melting and fusing together part of the powder or particles of a layer of material, then lowering the bed, adding a further layer of powder and repeating the melting and fusing process. The unfused powder around the fused material provides support so unlike some methods discussed above it may not be necessary to use support structures. Such methods include direct metal laser sintering (DMLS), electron beam melting (EBM), selective heat sintering (SHS), selective laser melting (SLM) and selective laser sintering (SLS). In view of the types of materials which are compatible with such processes (including metals and polymers), functional high strength materials can be manufactured.

Binder jetting methods are similar to powder bed fusion methods in that they use layers of powder or particulate material. However, conventional binder jetting methods differ from powder bed fusion methods in that the powder is not initially fused together but instead is held together with a binder which is jetted onto the structure from a print head. The binder may be coloured and the colour may be imparted to the powder thereby allowing colour 3D printing. Typically a binder is applied in a specific pattern to a layer of powder, and then the steps of applying a layer of powder and selectively applying binder are repeated.

In general, binder jetting entails the use of binder as a sacrificial material which is altered or removed in a post-processing step. This is because the adhesive binder typically imparts enough mechanical strength (termed “green strength”) to enable the structure to be self-supporting and maintain its shape as it is built up, and to withstand mechanical operations during manufacture, but not enough strength to be functional for the intended end use. Thus the structure is usually subsequently heated to remove the binder (de-binding process) and to fuse the build material together in a post-processing step to ensure that the product is fit for purpose which may include load-bearing or other applications.

Binder jetting is also referred to as the “drop-on” technique, “powder bed and inkjet 3D printing”, or sometimes just “3D printing”, though as summarised here there are many other different types of 3D printing. The binder used in binder jetting is generally liquid and is often referred to as “ink” in view of the inkjet application process.

One challenge with traditional binder jetting relates to porosity. The post-processing heat treatment step removes the binder and fuses the structure, but leaves significant porosity. This is partly due to the inherent packing densities which are possible with the particles of the powder bed, and partly due to the de-binding process. The de-binding process can also cause further problems, in particular shrinkage and contamination. The pores which remain can compromise mechanical properties. A further step of infiltration can be used to fill the pores, but this adds complexity and generally requires a different type of material so that the end product is generally weaker than an equivalent material made from a single material and is more difficult to recycle.

Yet further methods of 3D printing include lamination methods (wherein single sheets are formed and laminated together), and directed energy deposition (where powder is supplied to a surface and melted on deposition by e.g. a laser beam).

An Innovate UK assessment estimated the worldwide market for all additive manufacturing products and services to be worth $4.1 billion in 2014. Currently the sector has experienced a compound annual global growth rate of 35% over the last three years, driven by direct part production, which now represents 43% of the total revenue (“Shaping our National Competency in Additive Manufacturing”, 2012: https://connect.innovateuk.org). Future growth is forecast to be about $21 billion by 2020, which is expected to be driven by the adoption of additive manufacturing by the aerospace, medical devices, automotive and creative industries (“3D Printing and Additive Manufacturing State of the Industry,” W. A. Fort Collins, Editor 2014). Additive manufacturing has become a core technology within the field of high value manufacturing. Metals are the fastest-growing segment of the additive manufacturing sector, with printer sales growing at 48% and material sales increasing by 32% (Harrop, R. G. A. J., 3D Printing of Metals 2015-2025 Pricing, properties and projections for 3D printing equipment, materials and applications, IDTechEX, 2015.). Campbell et al (Campbell L, R. I., Bourell, D. and Gibson, I., “Additive manufacturing: rapid prototyping comes of age,” Rapid Prototyping Journal, 2012, 18(4): p. 255) have noted that the industry drivers for the development of additive manufacturing technology can be differentiated as:
Automotive—the ability to deliver new products to market quickly and predictably, significantly reduces overall vehicle development costs.
Aerospace—realisation of highly complex and high performance parts with integrated mechanical function, elimination of assembly features and enabling the creation of internal functionality (e.g. cooling etc.)
Medical—translation of 3D medical imaging data into customised solid medical devices, implants and prostheses.

Additive manufacturing is regarded as a disruptive technology that could be revolutionary and game changing, if barriers such as inconsistent material properties can be overcome. The present invention directly addresses this issue.

### THE PRESENT INVENTION

We have now developed a new method of binder jetting which uses new types of binder components.

From a first aspect the present invention provides a method of 3D printing comprising:
(i) providing a layer of a powder bed;
(ii) jetting a functional binder onto selected parts of said layer, wherein said functional binder infiltrates into pores in the powder bed and locally fuses particles of the powder bed in situ;
(iii) sequentially repeating said steps of applying a layer of powder on top and selectively jetting functional binder, multiple times, to provide a powder bed bonded at selected locations by printed functional binder; and
(iv) taking the resultant bound 3D structure out of the powder bed.

“Functional binder” herein means a binder which not only binds together the build material (conventionally the build material comprises the powder bed particles) but also becomes part of the build material. The present invention allows the production of end products which are functional products rather than prototypes. The functional binder is non-sacrificial: it contributes to the functional properties of the end product, e.g. properties of strength, rigidity, temperature-dependent behaviour, stability, inertness, corrosion-resistance, conducting, insulating or electronic properties, so that the end product may be suitable for use as a product, part or component in for example the automotive, aerospace or medical device industries. Such products, parts or components may for example be a components of vehicles or devices adapted to be used in or on the body.

The binder interacts with the surfaces of the powder bed particles so as to bind them together. The binder may do this directly or indirectly; in the latter case the binder may react during the jetting and/or deposition process to produce a more reactive species which then reacts with, and binds to, the surfaces of the powder bed particles.

The binder may for example be a metallic binder, a ceramic binder or a polymeric binder, or may be a mixture, e.g. a mixture of a metallic binder and a ceramic binder, or different metallic binders. The binder may bind together the powder bed particles with elemental metal or may result in a part of the end product which comprises a metallic or non-metallic compound or component. Thus the binder may result in the end product containing a metal, e.g. copper, nickel, titanium, aluminium or cobalt, amongst others, or an oxide and/or nitride and/or carbide, amongst others, of aluminium, silicon, beryllium, cerium, zirconium, or other metals or non-metals.

Where the binder is a metallic binder, we term the method “reactive metal jet fusion printing” (RMJF printing).

In the present invention, the binder used is a functional (e.g. metallic) binder; the binder infiltrates into the voids between the powder-bed particles in situ; and the powder-bed particles are fused in situ by application of the binder. The latter is due to the reaction with the functional binder and may also be facilitated by carrying out the process on a powder bed at a higher temperature than is conventional (conventionally, in binder jetting methods, powder beds are not heated). Without wishing to be bound by theory, chemical and physical processes are involved in forming the build material. The binder formulation may undergo a chemical transformation to for example result in a metal which physically fuses with the surrounding powder bed. The physical process may involve adsorption, diffusion and/or melting depending on the powder bed temperature.

The functional (e.g. metallic) binder contrasts with organic adhesive binders which have commonly been used hitherto. The present invention allows the ink to be used as a means of incorporating metal or ceramic into the structure. The metal or ceramic remains in the end product even if a post-processing step of higher temperature sintering is carried out. This contrasts with, and brings advantages with respect to, the use of sacrificial binders in the prior art.

It should also be noted that the present invention relates to the preparation of functional components or parts rather than mere prototypes. Binder jetting has been used in rapid prototyping: it enables 3D models to be produced easily. Such 3D models are not functional—their purpose generally relates to their appearance.

The infiltration of the binder into the voids between the powder-bed particles in situ differs from the conventional application of a binder which merely adhesively secures the powder bed layers. In the latter, significant porosity remains and this can lead to shrinkage or may require an infiltration procedure to be carried out in a post-processing step. In the present invention, the in situ infiltration results in a simpler process and enables reliable manufacturing of structures whilst addressing shrinkage issues.

Optionally, the extent of infiltration may be such that the residual porosity by volume of the product prepared by the method of the first aspect, before post-processing, may be no greater than 30%, or no greater than 20%, or no greater than 10%, or no greater than 5%, or no greater than 1%. In comparison, the achievable density in a conventional powder bed is of the order of 60% due to constraints on packing densities, so that conventional residual porosities are of the order of 40%. An extensive level of infiltration may be achieved by the metal binder conformally coating the particles of the powder bed at a surface level. The binders fill, or partially fill, the interstices between the powder bed particles. The binders may contain molecular components which enable surface-driven reactions to bring about chemical fusing, in contrast to the binding provided by conventional binder jet printing.

The porosity may be measured by computed tomography (CT), e.g. according to the method described in Mattana et al, Iberoamerican Journal of Applied Computing, 2014, V. 4, N. 1, pp 18-28 (ISSN 2237-4523).

The in situ fusing (e.g. joining, aggregation or bonding) of the powder particles with the metal of the binder brings further advantages compared to the use of a sacrificial adhesive binder; in particular the green strength of the material is enhanced, and composite and a wider range of tailored structures can be prepared.

Optionally one or more further step of post-processing may be carried out. In particular, the product may be heat-treated to consolidate and further strengthen, e.g. fuse, the structure. This may be done either after the application of each layer or after the entire structure has been built. The heat treatment step may be carried out at a temperature suitable for the material being used. For example, in some cases, it is beneficial to carry out a heat treatment step at a temperature towards, but not exceeding, the melting point of the material, e.g. steel 1100-1300, aluminium alloys 590-620, copper 750-1000, brass 850-950, bronze 740-780° C. It should be noted that this is a heat treatment step in contrast to the chemical process which occurs on application of the binder to the powder bed particles.

Thus the present method facilitates the preparation of dense, optionally substantially fully dense, functional, 3D printed parts and in particular is a step forward with regard to metal additive manufacturing and ceramic additive manufacturing.

Hitherto, only the powder bed fusion (PBF) technologies, such as selective laser melting (SLM), and more recently electron beam melting (EBM), have made significant inroads into the functional metal part market. These fusion based technologies, although impressive, have a number of problems, some related to the sub-optimal microstructure and others to scalability. The scalability has led to a limit on the size of objects that can be produced, lengthy manufacturing times, relatively high costs, problems with residual stress, and increasing difficulties with production as the size of the part increases. These problems have restricted SLM and EBM technologies to smaller, high added value-parts, and it is difficult to see how the technology can be scaled while controlling or reducing costs.

The present invention in effect combines the flexibility and agility of the laser powder melting techniques with the low cost of older powder bed print technologies.

The present invention benefits from some advantages of the binder jetting process compared to the powder bed fusion processes such as SLM and EBM (including: no support structures being required during the forming process, much higher layup speeds, ease of scaling and lack of internal stresses). At the same time the present invention addresses an Achilles' heel of known binder jet technology in that it infiltrates the pores with metal or ceramic binder which makes the products suitable for use as functional components, and avoids using weak binders which can lead to the parts sagging during post processing.

To highlight some advantages of the present invention it is instructive to consider some known comparative processes.

For example, binder jet company ExOne employ an aqueous-based binder ink, which strategically drops binder onto the powder bed, to form complex 3D metal “green” parts. The residual porosity of the green parts is then reduced by infiltration of molten metal using post-processing, hot isostatic pressing. In this instance, the infiltration process (>1100° C.) requires the use of a bronze filler. Each component therefore contains two alloys which renders it weaker than a conventional part and the part is more difficult to recycle. There is a tendency for the parts to shrink during these processes and therefore the parts need to be produced oversize initially, to allow for shrinkage. This shrinkage occurs because of the loss of the sacrificial binder leaving pores that are then consolidated during sintering. Predicting the likely shrinkage is difficult for complex parts. Attempts to overcome the shrinkage problem have been developed, including the work carried out by Bai and Williams (Bai, Y. and C. B. Williams, Rapid Prototyping Journal, 2015, 21(2): p. 177) who claimed the first binder jetting of complex 3D copper components that did not need infiltration. A thermosetting polymer binder was used to process a range of different sized copper powders (15.3 μm to 75.2 μm average diameters); after sintering in hydrogen/argon, a density of 78% (of theoretical density) was achieved, however an associated shrinkage of 37% was still observed for this approach. Also, Sasaki et al, from Ricoh Ltd, recently developed a novel binder process whereby the metal powders were coated in a 100 nm layer of water-soluble glue, which was then activated by jetting a water based ink onto the powder bed (Takafumi Sasaki, H. I., Takeo Yamaguchi, Daichi Yamaguchi, “Coated Powder Based Additive Manufacturing using Inkjet Technique”, Printing for Fabrication, 2016). Cross-linking then occurred to harden the parts. Although processing time was reduced due to less binder requirement, the parts were still weak, particularly in the build direction, making large parts difficult to handle.

In contrast the present invention significantly improves 3D binder printing by the use of an in situ infiltration process which effectively binds metal or ceramic powders, layer-by-layer, to manufacture 3D parts while filling the pores between the particles with functional metal or functional ceramic rather than a mere binder. The lack of a sacrificial binder ink enables parts with reduced shrinkage and higher densities. The present invention results in less waste and fast, economical, industrially relevant, 3D printing.

The binder of the present invention is a material which may be applied by a jetting process to result in a metal, alloy or compound bound to the surfaces of the powder particles in the powder bed. As discussed above, the binder is a functional binder, and may for example be a metallic binder or a ceramic binder. The binder may be in the form of a compound, salt or reagent, and may be in a carrier medium (e.g. a solvent), and the formulation may also comprise other components e.g. co-reagents (which may for example facilitate the conversion of compounds to elemental metals), other particles, and rheological agents to facilitate jetting, amongst other components.

The binder may comprise a molecular precursor of a metal or alloy, for example an organometallic material. The organometallic material may be a compound or complex which can react in situ to result in a metal or alloy bound to the surface. The material may be referred to as a reactive organometallic ink because it is printed onto the powder bed and reacts with the particulate material in the exposed powder bed layer.

Thus, whilst the present invention is applicable to a range of functional binders, one important class is metallic binders. Metallic functional binder inks may contain reactive metal compounds, for example metal halides or metal salts, and amongst the most useful of reactive metal compounds are organometallics. Reactive organometallic (ROM) material undergoes reaction to lose ligands and change to elemental metal and bind to the particles of the powder bed.

Optionally the binder composition may comprise, in addition to a component which reacts at the molecular level (e.g. ROM), nanoparticles e.g. metal or ceramic nanoparticles. Optionally it may further comprise microparticles, e.g. metal or ceramic microparticles.

The metallic or ceramic binders (or inks) are capable of chemically fusing metal powders through a chemical transformation or conversion. During this process a metal adlayer or ceramic adlayer joins the powder bed particles and any filler particles. This is analogous to joining parts using a molten solder.

Optionally the metal or ceramic composition used in the present invention may have a size-distribution ranging from the molecular to nanoparticle through to the microparticle size or any mixture thereof. The purpose of having a range of different particle sizes is to achieve extensively or fully densified microstructures. Thus, while reactive materials e.g. organometallic (ROM) materials result in conformal coating of the powder bed particles at the surface level, nano- and/or micro-particles fill the bulk of the voids or interstices. Therefore, optionally, the functional binder may comprise at least two components: a reactive material and a nanoparticulate and/or microparticulate material. Optionally the binder may comprise at least three components: a reactive material; a nanoparticulate material and a microparticulate material.

Thus the skilled person will understand that a spectrum of particle sizes should be used in the binder (which may for example range from molecular materials to nanoparticulate materials to microparticulate materials), to enable the space and interstices between the powder bed particles to be effectively filled. The most effective distribution of particle sizes to be used is preordained by the nature of the components making up the powder bed. The present inventors have recognised that, for any particular desired final material, a suitable matrix for the powder bed can be chosen, and that this then predetermines the distribution of particle sizes of the “ink” which will be appropriate to produce a fully-filled, fully-functional material.

By nanoparticulate is meant that the particle size is on average within the ranges 1 to 100 nm, or 5 to 100 nm, or 1 to 50 nm, or 1 to 20 nm, or 1 to 10 nm, or 2 to 8 nm, or 3 to 7 nm, or about 5 nm).

By microparticulate is meant that the particle size in the ink is on average within the ranges 0.1 to 10 microns, or 0.1 to 5 microns, or 1 to 5 microns, or 1 to 3 microns.

Thus it may be that the binder composition may comprise three components which, along with the powder bed particles, form the build material: a functional binder fraction, a nanoparticulate fraction and a microparticulate fraction. It may be that the functional binder fraction forms 0.1-10%, e.g. 0.5-8%, e.g. 0.7-2%, e.g. 0.8-1.2%, e.g about 1%, of the volume of the product. It may be that the nanoparticulate fraction and the microparticulate fraction together form 10-50%, e.g. 20-45%, e.g. 30-40%, e.g. 35-40% of the volume of the product. It may be that the ratio of nanoparticulate to microparticulate fraction in the product, by volume, is between 10:1 and 1:10, e.g. between 5:1 and 1:5, e.g. between 2:1 and 1:2, e.g. between 10:1 and 1:1, e.g. between 5:1 and 2:1, e.g. between 1:1 and 10:1, e.g. between 2:1 and 5:1.

The skillset of those working in 3D printing has generally not included detailed chemistry expertise. The inventive approach described herein arises in part from an understanding of how to use chemical components to interact to facilitate a step change in binder jetting efficacy.

From further aspects the present invention provides functional binder compositions used in the method of the present invention.

The inks infiltrate the porosity (typically about 40% porosity) in the powder bed lay-up. The infiltrated material may optionally comprise up to 20% by volume of reactive binder (e.g. ROM) with the balance being comprised of particles, other components and carrier. Together these components act as an infiltrating metallic or ceramic binder to hold the 3D part in a green state until it can be subsequently consolidated by heat treatment. By filling the powder lay-up with metal or ceramic binder the final porosity, distortion and shrinkage of the finished part are reduced.

Metals printed in accordance with the present invention include copper, nickel, titanium, aluminium and colbalt. Ceramics printed in accordance with the present invention include alumina and other materials including oxides and/or nitrides and/or carbides, amongst others, of aluminium, silicon, beryllium, cerium, zirconium, or other metals or non-metals. Cermets and oxide dispersion strengthened materials may also be produced. The present invention allows the production of materials which have active material parts, e.g. shape memory alloys, piezoelectric materials, etc.

In the case of metallic binders, optionally the present invention utilises volatile metal precursor (reactive organometallic (ROM)) compounds), developed for chemical vapour deposition processes, as the basis for ink formulations. We have previously reported the synthesis and characterisation of a family of copper (I) metal precursors based around cyclopentadienyl and isocyanide ligands. These have been injected onto heated substrates to form copper metal films in reducing environments (Willcocks, A. M., et al., “Tailoring Precursors for Deposition: Synthesis, Structure, and Thermal Studies of Cyclopentadienylcopper(I) Isocyanide Complexes,” Inorganic Chemistry, 2015. 54(10): p. 4869-4881). We have used the same approach to inkjet print conductive silver metal films (Black, K., et al., “Silver Ink Formulations for Sinter-free Printing of Conductive Films,” Sci. Rep., 2016. 6: p. 20814) exploiting silver reactive organometallic precursors developed earlier for atomic layer deposition. The ink jetting of a nickel binder allows the manufacture of nickel super-alloy metal composites, based on powder feed stocks, for example Inconel 625. Nickel binder inks also facilitate the manufacture of 3D nickel alloy parts. Previously nickel acetylacetonate has been used as a precursor for the deposition of metallic nickel via atmospheric-pressure chemical vapour deposition. In a reducing ambient, the metal could be formed at 250° C. and above (Maruyama, T. and T. Tago, “Nickel thin films prepared by chemical vapour deposition from nickel acetylacetonate,” Journal of Mat. Sci, 1993. 28(19): p. 5345-5348.). The printing of a titanium metal binder allows the processing of 3D components based on TiAl6V4, for example. An issue associated with printing titanium is its very high sensitivity to gettering of oxygen, hydrogen, carbon and nitrogen. To circumvent this inherent reactivity, titanium-anion “solutions” can be printed to counter the unwanted poisoning of the printed metal part. One option is the printing of Ti(N) or carbide solid solutions, with a nitrogen content of <5 at %. The ROM precursor in this case may be based on a volatile titanium amide (Ti(NR₂)₄, where R represents a volatile ligand) combined with a reducing ambient.

Aside from ROMs, other materials may be used including for example salts, halides, alkyls, alkylamides, silylamides, organophosphorous compounds, organosulphurous compounds, organohalides, ketones and aldehydes, amongst others.

The inks may incorporate certain concentrations of the ROM component (e.g. about 5-50%, e.g. 10-40%, e.g. 20-30%, w/w) combined with certain loadings of metal micro- and nano-particles (e.g. about 10-60%, e.g. 20-50%, e.g. 30-40%, w/w). The melting temperature of very small nanoparticles is typically suppressed compared with the bulk, because the relief of the very high surface energy: volume ratio provides the thermodynamic driving force for melting or sintering. Optionally further components may be present, for example to control the reactivity of metal nanoparticles towards unwanted reactions (e.g. oxidation) before they can be incorporated into the 3D metal part. The use of pre-treatments can “cap” or encapsulate the nanoparticles in a protective layer to stop oxidation. Optionally ionic surfactants (e.g. Brij™ or Tween™) may be used to deliver metallic fillers into the porosity left by the feedstock powder. For larger micron-scale filler metal particles encapsulation is generally not necessary; however optionally the surface passivation layers on these particles may be reduced via a range of reducing pre-treatments. Optionally encapsulation may be used to reduce the extent of unwanted native oxide into the RMJF 3D parts. Optionally viscosity modifiers and surfactants may be used to inhibit particle agglomeration in order to suspend the metal particulates in the ROM solutions.

Some examples of materials to which the present invention is applicable include aluminium and its alloys, shape memory alloys, oxide strengthened alloys, tungsten and tantalum alloys, steels, magnesium materials, ceramics and glasses. For example, magnesium can be made fireproof or corrosion-resistant by application of a surface matrix surrounding the powder.

Any suitable material may be used as the powder bed particles including those which are conventional used in powder beds. These include metals and ceramics, or mixtures thereof.

The binder material may be the same as the powder bed material or may be different, depending on the required properties and intended applications of the end product.

From further aspects the present invention provides 3D printed products obtained or obtainable by the method of the present invention. These are distinguishable from products made by other methods because of their properties, for example their porosity and lack of contaminants or sacrificial binder residue.

The present invention allows the preparation of products which have properties suitable for their function.

Because the binder used in the present invention is not a sacrificial binder and becomes part of the build material, the resultant product can exhibit improved properties structurally (e.g. strength or fatigue resistance), in terms of conductivity (electrically or thermally), or in other ways. Without wishing to be bound by theory, the present invention ameliorates the flaws in the product due to cracks and porosity thereby improving the mechanical properties.

For example, it may be that a product made in accordance with the present invention has an ultimate tensile strength of greater than 30 MPa, greater than 50 MPa, greater than 100 MPa, greater than 200 MPa, greater than 500 MPa, greater than 1,000 MPa or greater than 10,000 MPa. This may be parallel to the layers formed in the process, or perpendicular, or both.

It may be that a product, component, or part made in accordance with the present invention is an automotive part, an aerospace component, an engineering component, a structural component, a medical device, an implant or component thereof or a prosthesis or component thereof.

It may be that the product has a porosity of less than 10%, or less than 5%, or less than 1% of the bulk volume.

The inkjet binder printer used may be based on TTPs “Vista” technology print heads.

The binder jet printer is capable of printing metallic functional binders for multiple materials and layering metal powder feed stocks.

Optionally the binder printing system incorporates print heads that are capable of jetting micron-sized particles. This binder printing system enables flexibility in the use of a range of binder inks and produces a print system that is capable of building complex 3D components beyond what is currently feasible using known procedures.

From a further aspect the present invention provides apparatus for carrying out the method of the present invention.

The skilled person will understand that the different components of the binder may play different roles.

The nanoparticulate material may allow the sintering temperature to be reduced and plays a role in reducing porosity. It becomes part of the build material (i.e. is non-sacrificial).

The microparticulate material also plays a role in reducing porosity, at a different level. It becomes part of the build material (i.e. is non-sacrificial).

The ROM or other molecular material may help carry the particulate material to facilitate jetting, may bind the powder bed together, and converts to a material (e.g. metal or ceramic) which becomes part of the build material (i.e. is non-sacrificial).

Thus the conformal coating and reaction facilitated by the ROM or other molecular material, in combination with the further space-filling provided by the other components, and the sintering to produce a fully-filled, fully-functional, material, bring about considerable advantages compared to disclosures in the prior art. Waste and burn-off of materials are avoided, and the product has improved properties.

Alloys and other composite materials may be made by for example using a component (e.g. the microparticulate component or alternatively/additionally one of the other components) which is different to the powder bed material.

Further functionalisation may be brought about by for example using functionalised nanoparticles (or functionalised other components) to embed other properties into the final material.

The present invention will now be described in further non-limiting detail and with reference to the drawings in which:

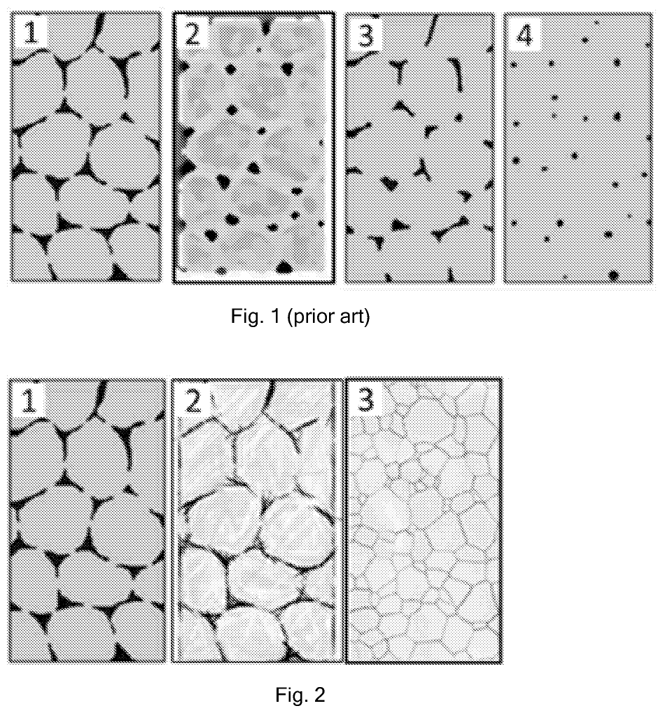

FIG. 1 shows schematic representations of material produced during stages of a conventional binder jet printing process; and

FIG. 2 shows schematic representations of material produced during stages of a process in accordance with the present invention;

The left hand panel (“1”) of each of FIGS. 1 and 2 shows a representation of a cross-section of part of a powder bed before binder jetting has taken place. It can be seen that there are significant voids between the particles.

Subsequent stages of a conventional binder jet printing process are shown in panels “2”, “3” and “4” of FIG. 1. “2” shows the product after the printing of sacrificial binder; “3” shows the product after sintering and removal of binder; “4” shows the product after a post-processing infiltration step.

In contrast, “2” in FIG. 2 shows the product after printing of metallic functional binder and simultaneous infiltration, in accordance with the present invention; and “3” shows the sintered densified end product in which no significant pores are visible.

In order to produce parts, it is necessary to deposit, layer by layer, the powder bed and to deliver ink formulations onto that bed in a controlled manner. This requires a powder bed mechanism similar to commercially available systems but with bespoke hardware and firmware to give full control over the process. The print-head jetting system is designed to give full access to control the ink jet print head system. In some embodiments the print-heads use TTPs “Vista” technology which uses a mechanical ejection process cable of delivering large sedimenting particle loaded inks and which can print inks that cannot currently be printed by commercially available industrial inkjet heads.

The powder bed may include a heating system that can heat the bed, with the maximum bed temperature likely to be <350° C., for example 50-350, e.g. 100-300, e.g. 150-250° C. Elevated bed temperature may be achieved by the use of a heater system under the bed or by radiant heaters above the bed, the objective in both cases being to activate the reactive binder (e.g., in the case of ROMs, to drive off the ligands from the ROM active part of the ink) and optionally to sinter the nanoparticles in the nano-component of the ink. This produces a fully-dense-high-strength “green” part, which can then be heat treated to create the correct final microstructure for functional use. Thus the moderate temperature at this stage fuses the nanoparticles and enables the reactive binder to release elemental metallic coating, whereas the post-processing heating fuses the larger microparticles.

Optionally the method lays metal powders with 25 μm precision, using a hopper-feed and wiper blade mechanism, which are designed to operate up to the maximum powder bed temperature. The print head and powder bed may be housed in a controlled environmental chamber (N₂ or Ar) to minimise atmospheric contamination and vent unwanted, noxious by-products. The system may be automated and run under computer control with a suitable build volume (e.g. 250×250×250 mm).

1. A method of 3D printing comprising:
providing a layer of a powder bed;
jetting a functional binder onto selected parts of said layer, wherein said binder infiltrates into pores in the powder bed and locally fuses particles of the powder bed in situ;
sequentially repeating said steps of applying a layer of powder on top and selectively jetting functional binder, multiple times, to provide a powder bed bonded at selected locations by printed functional binder; and
taking the resultant bound 3D structure out of the powder bed.

2. A method as claimed in claim 1 further comprising a subsequent step of heat treatment either inter-layer or post-build to further fuse the 3D structure.

3. A method as claimed in claim 1 wherein the functional binder comprises a metallic binder.

4. A method as claimed in claim 3 wherein the metallic binder comprises an organometallic material.

5. A method as claimed in claim 4 wherein the organometallic material is a copper metal precursor, for example comprising cyclopentadienyl and/or isocyanide ligands.

6. A method as claimed in claim 4 wherein the organometallic material is a nickel metal precursor, for example nickel acetylacetonate.

7. A method as claimed in claim 4 wherein the organometallic material is a titanium metal precursor, for example a titanium amide.

8. A method as claimed in claim 1 wherein the functional binder comprises a ceramic binder.

9. A method as claimed in claim 1 wherein the binder further comprises metallic or ceramic nanoparticles with sizes within the range of 1 to 100 nm.

10. A method as claimed in claim 1 wherein the binder further comprises metallic or ceramic microparticles with sizes within the range of 0.1 to 10 microns.

11. A method as claimed in claim 1 wherein the powder of the powder bed comprises metallic or ceramic particles.

12. A method as claimed in claim 1 wherein the functional binder is jetted onto the powder bed at a temperature within the range of 50 to 350° C.

13. A functional binder composition for binding particles of a powder bed, wherein the binder comprises:
(i) an organometallic material;
(ii) metallic or ceramic nanoparticles with sizes within the range of 1 to 100 nm; and
(iii) metallic or ceramic microparticles with sizes within the range of 0.1 to 10 microns.

14. A 3D printed product obtainable by the method of claim 1.

15. A 3D printed product comprising fused particles of metal and/or ceramic infiltrated with binder-jetted fused metal and/or ceramic.

16. A product as claimed in claim 14 which is a part or component of a vehicle or of a medical device, implant or prosthesis.

17. Apparatus for carrying out the method of claim 1.
